(19) United States
(12) Patent Application Publication
(10) Pub. No.: US 2026/0033967 A1
(43) Pub. Date: Feb. 5, 2026

(54) INTRAVASCULAR NERVOUS SYSTEM INTERFACE, APPARATUS AND METHOD FOR USING THE SAME

(71) Applicant: THE TRUSTEES OF COLUMBIA UNIVERSITY IN THE CITY OF NEW YORK, New York, NY (US)

(72) Inventors: Eleonora Francesca SPINAZZI, Teaneck, NJ (US); John William STANTON, New York, NY (US); Edward Sander CONNOLLY, JR., New York, NY (US); Kenneth L. SHEPARD, New York, NY (US); Sean D. LAVINE, Harrington Park, NJ (US)

(73) Assignee: THE TRUSTEES OF COLUMBIA UNIVERSITY IN THE CITY OF NEW YORK, New York, NY (US)

(21) Appl. No.: 19/301,758

(22) Filed: Aug. 15, 2025

Related U.S. Application Data
(63) Continuation of application No. PCT/US2024/015937, filed on Feb. 15, 2024.
(60) Provisional application No. 63/445,867, filed on Feb. 15, 2023.

(51) Int. Cl. A61F 2/90; A61B 5/00; A61B 5/25; A61B 90/00; A61F 2/48; A61F 2/95; A61F 2/966

(52) U.S. Cl. CPC A61F 2/90; A61B 5/25; A61B 5/6862; A61B 5/6876; A61B 90/39; A61F 2/481; A61F 2/482; A61F 2/966; A61B 2090/3966; A61B 2560/0468; A61B 2560/063; A61F 2002/9534; A61F 2250/0002; A61F 2250/0067; A61F 2250/0098

(57) ABSTRACT

An exemplary device (e.g., an intravascular prosthesis) can be provided which can comprise an expandable configuration (e.g., a housing) configured or structured to be inserted within a luminal biological structure (e.g., a blood vessel). The expandable configuration can include at least one self-expanding system which is (i) a wire system and/or a mesh system, and (ii) configured to include a plurality of apical connectors, a subset thereof connecting to a medial structure. The expandable configuration can also include at least one flexible interconnect system configured or structured to house (i) at least one integrated circuit, (ii) at least one electrode, and (iii) at least one transducer. Alternatively or additionally, the expandable configuration can comprise a medial structure connected to one or more of the apical connectors, and which is be dissolved within the tubular biological structure to nontoxic species over a controlled time frame. Exemplary methods can also be provided for installing such exemplary intravascular prosthesis within a luminal biological structure.

### CROSS REFERENCE TO RELATED APPLICATION(S)

This application relates to and claims priority from International Patent Application No. PCT/US2024/015937 filed on Feb. 15, 2024 which was published as International Publication No. WO 2024/173642 on Aug. 22, 2024, and from U.S. Provisional Patent Application No. 63/445,867, filed on Feb. 15, 2023, the entire disclosures of which is are incorporated herein by reference.

### FIELD OF THE DISCLOSURE

The exemplary embodiments of the present disclosure are directed to the field of endoluminal blood vessel implants. In particular, the exemplary embodiments of the present disclosure relate to scaffolding system(s), apparatus, interface(s) and method(s) for providing structural support, deployment, sheathing, re-sheathing and/or retrieval of endovascular and trans-vascular electroceutical implants.

### BACKGROUND INFORMATION

Nervous system interfaces that can stimulate and record from tissue are revolutionizing the treatment of pharmacologically-resistant disease phenotypes (see, e.g., Ref. [1]). Despite widening applications, the implantation of devices for these purposes continue to be invasive procedures delivering large volume implants (>about 1000 mm³) with penetrating or cuff electrode leads placed around the peripheral nervous system target, such as, e.g., the Microtransponder Vivistim® device.

Conventional stents can be elongated tubular prostheses that can be typically used to support the endoluminal walls of a vessel. Recent advances in stenting technologies can provide clinicians with the ability to re-sheath stent structures so they can be optimally positioned inside the vessel. Furthermore, stent structures augmented with electrodes suitable for electrophysiology have been developed, providing access to stable, long-term, minimally invasive interfaces via vascular structures proximal to peripheral nerves (see, e.g., Ref. [2]). However once deployed, devices of this type can become permanently implanted, offering little recourse in the event of malposition, or an adverse reaction to the presence of the foreign device residing in the vessel.

Vascular filters, which are specifically filters designed for deployment in the inferior vena cava, can reduce the risk of clot migration and embolization into the cardiopulmonary vasculature by providing a mechanism to capture dislodged plaques or clots in the blood stream. These devices can be typically transiently implanted, e.g., designed to be placed for a short amount of time and therefore, are configured with a mechanism for removal and/or retrieval. The unique design of vascular filters, however, has not offered a mechanism or an arrangement for a transient repositioning of the device once it has been deployed in a vascular context, and devices of this type have found no further applications than for a clot retention. Specifically, re- sheathing, or restoring a partially deployed inferior vena cava filter back into its delivery mechanism can be particularly advantageous.

Thus, it may be beneficial to provide systems, devices, and methods for a re-sheathable and retrievable wireless nervous system implant that accesses nervous system tissue from the vasculature, overcoming at least some of the deficiencies described above.

### SUMMARY OF EXEMPLARY EMBODIMENTS

To that end, in order to address such deficiencies and others, scaffolding system(s), apparatus, interface(s) and method(s) according to the exemplary embodiments of the present disclosure for providing structural support, deployment, sheathing, re-sheathing and/or retrieval of endovascular and trans-vascular electroceutical implants are provided and discussed herein.

Provided herein is a summary of an exemplary implementation of the re-sheathable and retrievable intravascular nervous system interface in accordance with various exemplary embodiments of the present disclosure, and is not intended to limit or restrict the scope of the present disclosure.

The device according to an exemplary embodiment of the present disclosure can comprise a self-expanding metallic structure that can include a plurality of support struts and a plurality of connector elements. A subset of the supporting struts can be oriented in parallel with the vessel lumen, and can comprise the total length of the implant. The exemplary device can include one or more apical connector elements provided between struts placed on both the proximal side and the distal side of the implant, thereby creating a circumferential zig-zag pattern. For example, when the connector elements on the proximal side of the implant can connect, e.g., only to other luminally oriented struts, the connector elements on the distal end of the implant can include a third strut connection to a ring suspended in the center of the vessel. The ring can provide, for example, an interconnect mechanism for a delivery system to disconnect from during implantation, and a capture point for a retriever system to affix to during retrieval.

For example, the metallic structure can be compressed to a reduced radius, and placed in a sheathable delivery vehicle, allowing it to navigate through relatively small and curved vessels to a deployment target. Upon reaching the desired treatment site, the exemplary device can be unsheathed, facilitating the compressed stent to self-expand radially outward to the vessel extents. In its unsheathed state, the exemplary device can be fully deployed, or transiently re-sheathed back into the delivery vehicle. If the clinician decides to re-sheath the exemplary device, the clinician can draw the implant into the delivery vehicle and subsequently reposition, rotate, and/or remove the device from the patient either partially or entirely. When the clinician is satisfied with the position and orientation of the unsheathed implant, the exemplary device can be placed into a fully deployed state via the dissociation of the implant from the delivery vehicle. This separation can be achieved through an electrolytic detachment or a mechanical disconnection of an interconnect facilitated by a mechanically manipulatable element in the delivery vehicle controlled externally by the clinician.

Upon deployment, the struts can hold the polyimide scaffolding in place flush against the vessel walls on one end, and on the other end come to a midpoint in the center of the vessel held together with bioabsorbable polymeric ring fragments comprised of a bioabsorbable polymer, providing certain advantages relative to mostly-or all-metal center rings. For example, such exemplary device can facilitate a natural decomposition of connecting elements of the center ring into non-toxic, inert chemical species over a finite and predetermined time window. Over this exemplary period, the bioabsorbable ring can gradually degrade, while the exemplary system can endothelialize and/or integrate itself into the vessel context. Upon a dissolution, e.g., the ring no longer holds the struts in the center of the vessel, and the implant can fully endothelialize and/or integrate with the rest of the vascular context. This can provide a time window over which the exemplary system can safely integrate into the parent vessel wall without the risk of migration/embolization, and can be readily explanted, if necessary.

The exemplary system can be designed and/or configured to endothelialize and remain in the patient permanently. The exemplary system can also be designed and/or configured to provide for the explant of the exemplary system by the clinician in the case of an adverse reaction, the desire to suspend treatment, and/or any other clinically relevant reason. Prior to the dissolution, the exemplary ring can provide a structure that can be gripped by a retriever system, thus facilitating the implant device to be re-sheathed into the retrieval microcatheter and explanted from the patient entirely. To facilitate an explant, an exemplary retriever system can be introduced and/or provided such that a hook or gripping mechanism affixes to the ring in the center of the vessel, and a tubular case is extended in the luminal direction over the ring support struts. As the tubular structure extends over the plurality of struts holding the center ring in place, the luminal support struts of the implant can fold inward, thus radially compressing the stent down to a total cross section that is compatible with vascular navigation. The exemplary retriever system and the exemplary compressed device, which are in this manner mechanically connected, can be removed from the patient either partially or entirely.

The exemplary device and system can include a flexible interposer system/configuration that can facilitate a connectivity between one or more electrodes, one or more application specific integrated circuits (ASICs), one or more energy storage elements, and/or one or more transducer elements. In certain exemplary embodiments of the present disclosure, such exemplary interposer system can be or include a flexible circuit board, and the board does not need to be a uniform sheet. To affix the flexible circuit board on the extents of the plurality of connector elements, the exemplary system can include circuitous electrodes, and can interconnect in, e.g., a zig-zag pattern when maximally compressed, such that as the self-expanding wire mesh can radially expand the electrodes and interconnect in an open up accordion style, e.g., fanning out with the extents of the metal scaffolding. The flexible electrode board can be affixed to the wire mesh structure, for example, through the use of biocompatible adhesives, via through-hole connections around one or more of the wire-mesh struts, or through the inclusion of an affixing mount point on the scaffolding itself.

In certain exemplary implementations of the implantable device according to exemplary embodiments of the present disclosure, the stent can be coated with a drug-eluting polymer configured to gradually release a therapeutic pharmacological agent. This agent can assist with the mitigation of restenosis, platelet activation, and/or thrombogenesis. The exemplary coating and/or pharmacological agent(s) can be configured or designed to have a well-defined release pharmacokinetics and loading capacity.

In some exemplary implementations of the implantable device according to exemplary embodiments of the present disclosure, one or more asymmetric radiopaque fiducials can be fabricated on the implant, thus facilitating the deconvolution of the rotational orientation from the fluoroscopic imaging alone. This exemplary fiducial can be used to precisely place the exemplary system in the vessel, both in terms of a luminal position and/or an angular rotation.

According to further exemplary embodiments of the present disclosure, an intravascular prosthesis, which can comprise an expandable configuration that is configured or structured to be inserted within a luminal biological structure. The expandable configuration can comprise at least one self-expanding system which is (i) a wire system and/or a mesh system, and (ii) configured to include a plurality of apical connectors, with a subset thereof being connected to a medial structure. The expandable configuration can also comprise at least one flexible interconnect system configured or structured to house or provide therein or thereon (i) at least one integrated circuit, (ii) at least one electrode, and (iii) at least one transducer. The expandable configuration can also be further configured or structured to be deployed with a delivery vehicle into the luminal biological structure by (i) compressing the housing to form a compressed state, and (ii) deploying the expandable configuration at a location of a therapeutic relevance by expanding the housing from the compressed state to an expanded state at one or more extents of the luminal biological structure (e.g., a blood vessel, etc.)

According to yet another exemplary embodiment of the present disclosure, an intravascular prosthesis can be provided that comprises an expandable configuration that is configured or structured to be inserted within a blood vessel. The expandable configuration can comprise at least one self-expanding system which is (i) at least one of a wire system or a mesh system, and (ii) configured to include a plurality of apical connectors. The expandable configuration can further comprise a medial structure connected to one or more of the apical connectors. The medial structure can be configured to (i) facilitate a placement of the intravascular prosthesis into a patient, and (ii) be dissolved within the luminal biological structure to nontoxic species over a controlled time frame.

In additional exemplary embodiments of the present disclosure, the flexible interconnect system can be or include at least one flexible circuit board, and the flexible circuit board(s) can comprise polyimide and metal interconnects. The electrode(s) can spans a predetermined percentage of a dimension of the at least one flexible circuit board, such that (i) when compressed, the flexible circuit board(s) can maintain a substantially similar cross-sectional area as that of a mesh scaffolding, and (ii) when expanded in the blood vessel, the flexible circuit board(s) can span a predetermined percentage of a circumference of the luminal biological structure. For example, the electrode(s) can be configured to stimulate or record electrophysiological information for at least one biological portion provided in or at the luminal biological structure. Additionally or alternatively, the self-expanding system can include thereon or therein a pharmacological-eluting coating.

In still further exemplary embodiments of the present disclosure, the expandable configuration can be configured or structured to include an asymmetric fluoroscopic imaging fiducial. The transducer(s) can include a piezoelectric material that can be configured to provide telemetry and power to the integrated circuit(s) with ultrasound. The medial structure (e.g., a hub) can be configured to facilitate a retrieval of the intravascular prosthesis from a patient and/or be dissolved to nontoxic species over a controlled time frame, as well as be configured to facilitate a placement of the intravascular prosthesis into a patient. The self-expanding system(s) can include the medial structure, and/or be an expandable housing which can include the medial structure.

According to an additional exemplary embodiment of the present disclosure, a method can be provided for installing an intravascular prosthesis within a luminal biological structure. In this exemplary method, a tubular device can be provided into the luminal biological structure. The intravascular prosthesis (which includes an expandable configuration) can be extended through the tubular device via an implantation device. The expandable configuration can comprise at least one self-expanding system which is (i) at least one of a wire system or a mesh system, and (ii) includes a plurality of apical connectors.

Such expandable configuration can also comprise a medial structure connected to one or more of the apical connectors, and which is be dissolved within the tubular biological structure to nontoxic species over a controlled time frame. Alternatively or additionally, the expandable configuration can comprise at least one flexible interconnect system configured or structured to house or provide therein or thereon (i) at least one integrated circuit, (ii) at least one electrode, and (iii) at least one transducer.

The intravascular prosthesis can be pushed or otherwise placed into the luminal biological structure via the implantation device to be outside of the tubular device. In this manner, the self-expanding system opens by expanding the plurality of apical connectors to contacts inner walls of the luminal biological structure. Further, the implantation device can be disconnected from the intravascular prosthesis to implant the intravascular prosthesis, and the tubular device and the implantation device can be removed from the biological structure.

These and other objects, features and advantages of the exemplary embodiments of the present disclosure will become apparent upon reading the following detailed description of the exemplary embodiments of the present disclosure, when taken in conjunction with the appended claims.

### BRIEF DISCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is a diagram of an exemplary implantable intravascular prosthesis according to exemplary embodiments of the present disclosure;

FIG. 2 is a side cross-sectional view the exemplary prosthesis in an exemplary delivery vehicle according to exemplary embodiments of the present disclosure;

FIG. 3a is a flowchart for an exemplary implantation of the implantable intravascular prosthesis according to exemplary embodiments of the present disclosure;

FIG. 3b is a flowchart for an exemplary retrieval of the implantable intravascular prosthesis according to exemplary embodiments of the present disclosure;

FIGS. 4A-4F are a set of side cross-sectional progression view of a transition from the compressed state to the expanded state of the implantable intravascular prosthesis according to exemplary embodiments of the present disclosure;

FIGS. 5A-5D are illustrations of a set of side views of an exemplary electrolytic separation of the implantable intravascular prosthesis according to exemplary embodiments of the present disclosure from the delivery vehicle;

FIGS. 6A-6F are illustrations of a set of side views of the transition of the implantable intravascular prosthesis from implanted to explanted through a retrieval system according to exemplary embodiments of the present disclosure;

FIG. 7A is a cross sectional view of the asymmetric imaging fiducial according to exemplary embodiments of the present disclosure;

FIG. 7B is a perspective view of a representative asymmetric imaging fiducial design according to exemplary embodiments of the present disclosure;

FIG. 7C is an illustration of the mount point of the asymmetric imaging fiducial on the intravascular prosthesis according to exemplary embodiments of the present disclosure;

FIG. 8A is an illustration of a representative electronic interconnect board in the compressed state according to exemplary embodiments of the present disclosure;

FIG. 8B is an illustration of a representative electronic interconnect board in the expanded state according to exemplary embodiments of the present disclosure;

FIGS. 9A-9C are a set of illustrations of the attachment of the flexible electrode board on to the implant according to exemplary embodiments of the present disclosure; and

FIG. 10 is an illustration of the exemplary prosthesis after an erosion of the bioresorbable center ring according to exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

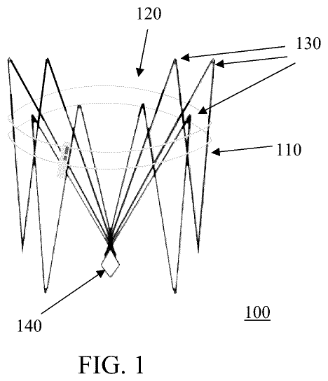

The present disclosure describes exemplary embodiments of a intravascular prosthesis that can comprise a retrievable/re-sheathable metallic scaffolding element that supports a flexible interposer. The exemplary flexible interposer can facilitate flexible electrodes, electronics, transducers, interconnect, telemetry, and/or energy storage elements. An exemplary mode of operation is shown in FIG. 1, which illustrates a diagram of an exemplary implantable intravascular prosthesis 100 according to exemplary embodiments of the present disclosure.

In the exemplary embodiment of the present disclosure, as shown in FIG. 1, the metallic scaffolding of the implantable intravascular prosthesis 100 can comprise a number (e.g., eight) structural elements 110 configured to align with the luminal dimension of a blood vessel in a zig-zag pattern around a circumference of the vessel. Such exemplary metallic structural elements 110 can be configured to extend from the circumferential extent to the center of the prosthesis. In one example, twelve (12) apical connector elements can be provided which can be configured to interface between one or more of these sixteen (16) total structural elements. In such example, eight (8) of the apical connectors can be placed or otherwise provided on the distal end of the exemplary prosthesis 100, and four (4) of the apical connectors are on the proximal end of the exemplary prosthesis 100, providing a structural continuity.

While—in this exemplary embodiment—the proximal apical connectors may only interface one or more (e.g., pairs of) circumferential structural elements, the distal apical connectors can be configured to connect a circumferential strut element to a strut element configured to extend from the circumferential extent into the center of the prosthesis. For example, all eight of the medial ends 130 of the center-terminating struts can be configured with one or more (e.g., two) radial connecting elements, forming a structural ring in the center of the prosthesis 100, e.g., a center ring 120. In alternative exemplary embodiments of the present disclosure, a varying number of support struts and corresponding number of connecting elements can be provided. For example, six, ten, etc. luminally-aligned struts can be used instead of eight struts, provided that the struts are configured to zig-zag around the circumference of the vessel with a corresponding number of apical/center connecting elements.

The expandable metallic structure can be composed of—at least partially or fully—a biocompatible material. Examples of elastic materials can include spring steels, stainless steel, alloy NP36N, Nitinol, Elgiloy, and similar metals and alloys. It is also possible to coat these materials with porous or textured surfaces for cellular ingrowth, or with non-thrombogenic agents, including but not limited to pyrolytic carbon, Teflon materials, heparin, hydrogels, silicones, polyurethanes, etc. The exemplary metallic struts can be treated such that pharmacological agents can be eluted therefrom. In the exemplary embodiment of the present disclosure, the metallic structure can comprise of a plurality of single struts, and alternative exemplary embodiments can utilize an expandable wire-mesh structure.

The exemplary metallic scaffolding of the implantable device 110 according to the exemplary embodiments of the present disclosure can have, e.g., two structural states, a radially compressed/contracted state and a radially expanded state. In the compressed state, the prosthesis is reduced to approximately thirty percent of the cross-sectional area of its radially expanded state. This can facilitate the prosthesis to traverse the vascular system to the desired interventional target site even through small diameter vascular pathways. The prostheses of the same or similar structural design with varying compressed/expanded radii can be manufactured or otherwise provided to facilitate a clinician to tailor an appropriately sized prosthesis with anatomical specificity on a per patient basis.

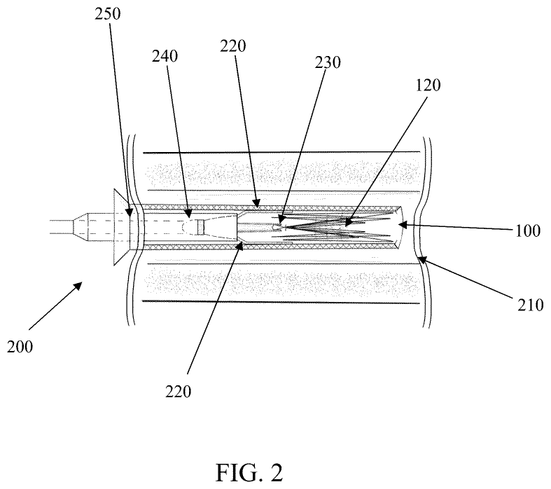

In the compressed state, the prosthesis 100 can be contained or provided inside a further system, device, or method according to additional exemplary embodiments of the present disclosure that can provide a mechanism to deliver the prosthesis in a controlled manner into a vascular context. The exemplary delivery mechanism or vehicle can be or include a tubular system that can be designed to carry the prosthesis to its interventional target site, holding the prosthesis in its radially compressed state. The exemplary delivery mechanism or vehicle can provide or include a mechanism for the clinician to facilitate the transition from the radially compressed to radially expanded state via a mechanical tether from the delivery mechanism/vehicle to the prosthesis which extends outside of the patient for clinician control. The exemplary device placed in the exemplary delivery vehicle 200 is depicted in the illustration of FIG. 2.

For example, the expanded state can have two further sub-states, a re-sheathable state, and a fully deployed state. In the re-sheathable state, the exemplary prosthesis 100 has been pushed out of the delivery vehicle 200, facilitating the device to expand outward to the vessel extents. In this state, the prosthesis 100 is still mechanically connected to the delivery vehicle 200 via the hub 140 of the prosthesis 100 and the connection configuration 230 of the delivery vehicle (e.g., which can be or include a hooking arrangement or any other connection device for the hub 140). In the re-sheathable sub-state, the prosthesis 100 can be pulled back into the delivery vehicle 200 by pulling the connector 240 through the tubular structure 210 via the pulling/pushing mechanism 250, thus returning to the radially compressed state. Such pulling procedure can also be assisted with the use of the clamps which 220 of the delivery device 200. In particular, the clamps 220 close upon pulling the prosthesis 100 away from the body via the pulling/pushing mechanism 250, and open when the delivery device (via the pulling/pushing mechanism 250) pushes the prosthesis 100 toward the body. It should be understood that the pulling/pushing mechanism 250 can be used to rotate the prosthesis 100 within the tubular structure, and also within he blood vessel/luminal structure.

This exemplary configuration can facilitate the clinician to move the prosthesis 100 along the luminal axis, as well as alter the rotation of the implant 100. The physician can modify the position and/or the rotational orientation of the prosthesis 100, for example, to optimally align the implant 100 with an external system, configuration or device that can have a functional dependence on the vector between the external element and the implanted prosthesis 100, or a sub-element of the implanted prosthesis 100. Alternatively or in addition, an element of the prosthesis 100 may have a positional or rotational dependence on an anatomic target for therapeutic intervention. Further, when the physician is satisfied with the placement of the prosthesis the physician may initiate a sequence to separate the implanted prosthesis from the delivery vehicle.

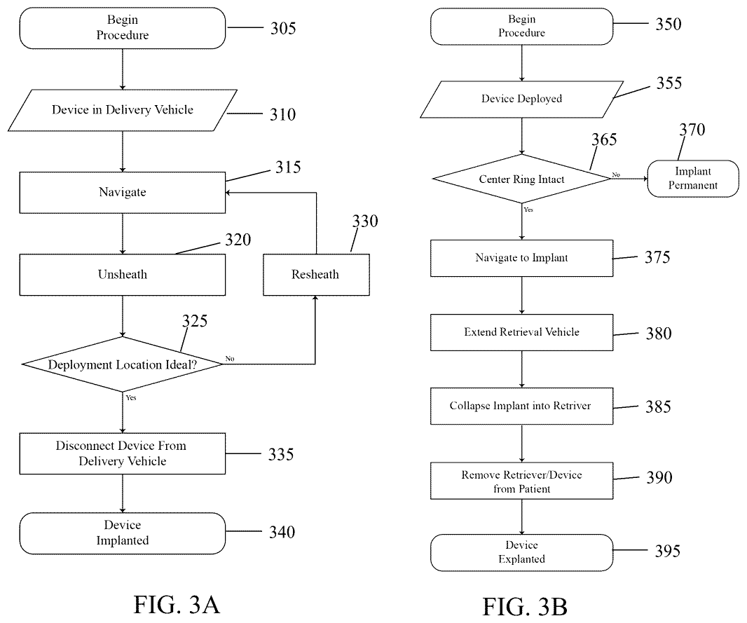

FIG. 3A shows an illustration of a method providing or facilitating a sequence of states the implant may experience during the implantation procedure according to one exemplary embodiment of the present disclosure. For example, in step 305, the procedure is initiated. In step 310, the prosthesis 100 is provided in the delivery vehicle 200, e.g., in a tubular structure 210 thereof. The prosthesis 100 is then navigated in step 315, and the prosthesis 100 is unsheathed from the tubular structure 210 in step 320. In step 225, it is determined in step 325 whether the deployment location of the prosthesis 100 is believed to be ideal or even appropriate. If not, the implantation procedure presheaths the prosthesis 100 in step 330, returns to step 315, and proceeds to repeated until the desire deployment location is reached. Upon reaching such desired deployment location, in step 335, the prosthesis 100 is disconnected from the delivery vehicle 200, and in step 340, the prosthesis 100 is implanted.

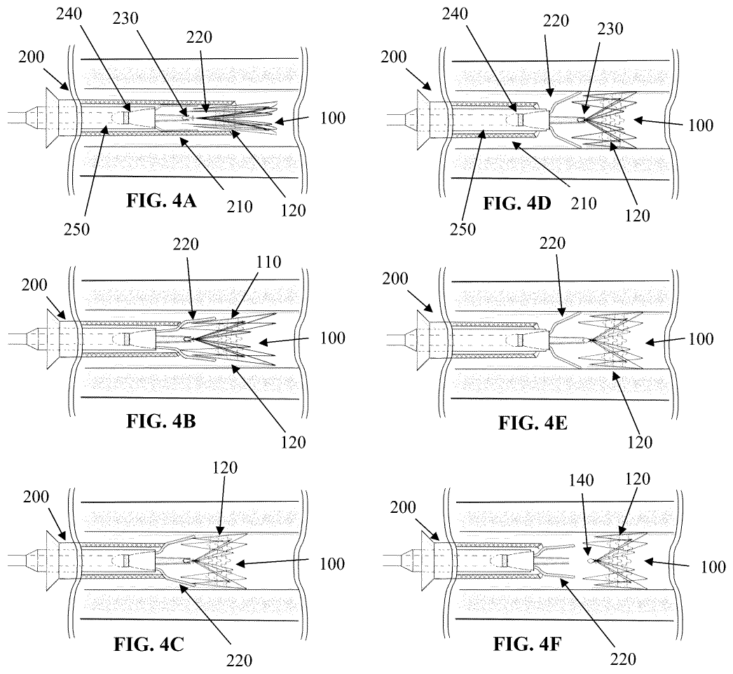

FIGS. 4A-4F illustrate a progression of the deployment of the exemplary device (e.g., the prosthesis 100) from the delivery vehicle 200 into the vascular context, according to an exemplary embodiment of the present disclosure. In particular, as shown in FIG. 4A, the prosthesis 100 that is connected to the delivery vehicle 200 is provided into a luminal structure (e.g., a blood vessel). In the initial configuration, the prosthesis 100 is mechanically connected to the delivery vehicle 200 via the hub 140 of the prosthesis 100 and the connection configuration 230 of the delivery vehicle, the clamps 220 are provide in the closed state, which cause the metallic structural elements 110 to be in a compressed state. To reach a desired location and/or orientation within the liminal structure, the pulling/pushing/rotational arrangement 250 and the connector 240 of the delivery vehicle 200 pushes, pulls and/or rotates the prosthesis 100.

Next, as shown in FIG. 4B, the pulling/pushing/rotational arrangement 250 and the connector 240 causes the clamps 220 to open, thereby causing the metallic structural elements 110 of the prosthesis 100 to decompress and partially open to impact the walls of the luminal structure. In FIG. 4C, the pulling/pushing/rotational arrangement 250 pushes the prosthesis 100 further into the luminal structure via the hub 140 and also causing the metallic structural elements 110 of the prosthesis 100 to be provided in a more opened position, so that the prosthesis 100 is embedded into the luminal structure. In FIGS. 4D and 4E, the pulling/pushing/rotational arrangement 250 causing the metallic structural elements 110 of the prosthesis 100 to be fully opened, and not be in contact with the prosthesis 100. In FIG. 4F, the connection configuration 230 of the delivery device 200 is disconnected from the hub of the prosthesis 100, thereby embedding the prosthesis 100 in the prosthesis 100, and at the same time removing the delivery device 200.

The center interconnected ring and/or hub 140 can be configured to provide a mechanical interconnect to further systems, devices, and methods according to the exemplary embodiments of the present disclosure that can facilitate the transition of the implantable prosthesis 100 from its compressed state to its expanded state, and vice versa. In the exemplary implementation, the interconnects of the hub 140 can be comprised or composed of a bioabsorbable material, either at least partially or fully. Examples of suitable bioabsorbable materials can include, but are not limited to, polylactic acid (PLA), polyglycolic acid (PGA), copolymer poly(lactide-co-glycolide) (PLGA), polydioxanone, polyanhydrides, trimethylene carbondate, poly(hydroxybutyrate), poly(g-ethyl glutamate), albumin, collagen, poly (ortho esters), polycyanoacrylate, polyphosphazenes, poly(a-hydroxy acids), poly(e-caprolactone), gelatin, alginate, starch, polysaccharides (e.g. cellulose, chitin, dextran), modified proteins (e.g. fibrin, casein), and/or copolymers, mixtures or combinations thereof. In further exemplary embodiments of the present disclosure, the hub 140 can be permanent, and/or comprised or composed of a similar metallic alloy as the stent struts.

In the exemplary implementation according to the exemplary embodiments of the present disclosure, the delivery vehicle 200 can be configured to affix to the hub 140 of the prosthesis 100 using a series of circumferential interconnect bands. These exemplary bands can be electrically conductive and are separated by an insulating spacer, thus forming, e.g., two distinct conductors. For example, one side of the interconnect bands can be interfaced to a power source through a conductive pathway along the length of the delivery system to its external end, forming an anode. While connected to the delivery vehicle 200, the opposing adjacent side of the interconnect band on the prosthesis can have a separate electrically distinct conductive pathway to the external end of the delivery system, forming a cathode. An electric current can be applied between the two interconnect bands of sufficient magnitude to electrolytically displace the insulating spacer. The patient's body can provide a conductive electrolyte between the anode and cathode while the interconnect is eroded, and, eventually fully disconnected. The exemplary delivery system can then be removed from the patient.

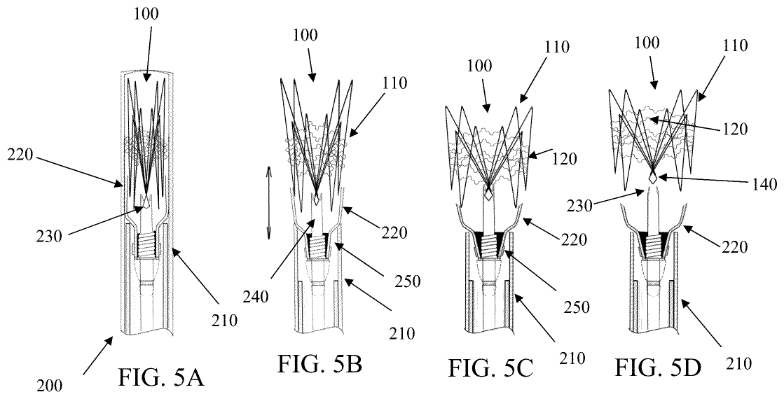

An exemplary procedure for disconnecting the prosthesis 100 from the delivery vehicle 200 is shown in the illustration of FIG. 5A-5D. In particular, as shown in FIG. 5A and also in FIG. 4A, the prosthesis 100 that is connected to the delivery vehicle 200 is provided into a luminal structure. The prosthesis 100 is mechanically connected to the delivery vehicle 200 via the hub 140 of the prosthesis 100 and the connection configuration 230 of the delivery vehicle, the clamps 220 are provide in the closed state, which cause the metallic structural elements 110 to be in a compressed state. In FIG. 5B, similarly to FIG. 4B, the pulling/pushing/rotational arrangement 250 and the connector 240 causes the clamps 220 to open, thereby causing the metallic structural elements 110 of the prosthesis 100 to decompress and partially open to impact the walls of the luminal structure. Then, as shown in FIG. 5C which is also similar to the illustrations of FIGS. 4D and 4E, the pulling/pushing/rotational arrangement 250 causing the metallic structural elements 110 of the prosthesis 100 to be fully opened, and not be in contact with the prosthesis 100. Finally, as shown in FIG. 5D which is similar to the illustration of FIG. 4F, the connection configuration 230 of the delivery device 200 is disconnected from the hub of the prosthesis 100, thereby embedding the prosthesis 100 in the prosthesis 100, and at the same time removing the delivery device 200.

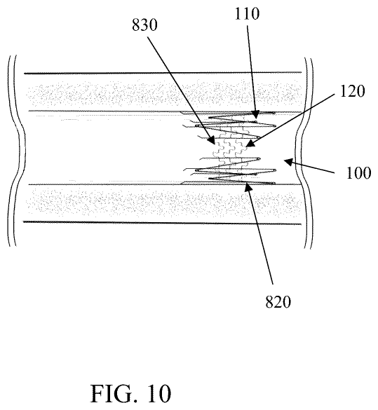

In the exemplary embodiment of the present disclosure, the hub 140 can be configured to have a well-defined degradation time course, such that it can maintain structural integrity for a clinically relevant window of time. After this defined exemplary time window, the hub 140 can, e.g., fully erode, and the centering struts can relax against the walls of the vessel, thus ultimately aligning with the luminal dimension of the vessel. As a result, the center ring 120 can be dissolved, and the implant 100 can be fully integrated into the vascular context in which it is deployed. The elimination of the hub 140 can also remove the mechanism through which the prosthesis is manipulated between its compressed and expanded states, thus the implant (e.g., the prosthesis 100) becomes a permanent fixture. The implant (e.g., the prosthesis 100) with the center ring 120 degraded is shown in the illustration of FIG. 10, e.g., without the hub 140.

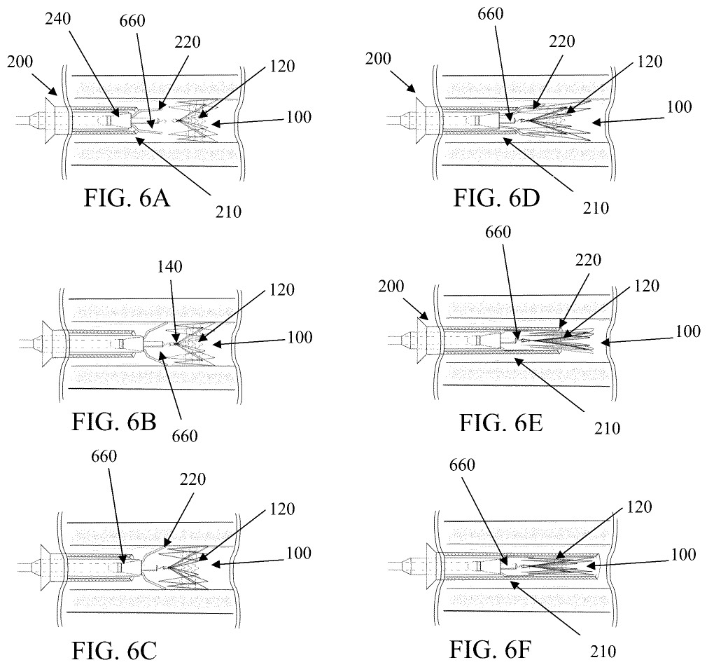

Prior to its dissolution, the hub 140 can provide an exemplary mechanism for a further retrieval system, device, and method according to the exemplary embodiments of the present disclosure to remove the implanted prosthesis 110 from its vascular context. According to the exemplary embodiments of the present disclosure, this exemplary retrieval system 200 can be or include a separate distinct tubular structure 210 that can be introduced to the vascular context adjacent to the implanted prosthesis 100 and manipulate the mechanical state of the implant (e.g., the prosthesis 100), providing a way for it to translate from the expanded/implanted state back to a compressed state. Ultimately, the exemplary retrieval system 200 encases the implantable prosthesis 100 in its compressed state facilitating for complete explant from the vascular context. The exemplary retrieval system 200 can comprise a tubular structure 210 to hold the explanted prosthesis 100, and a manipulatable hooking mechanism configured to mechanically affix to the hub of the implanted prosthesis. FIGS. 6A-6Fillustrate the exemplary progression of the explant of the exemplary device according to such exemplary embodiments of the present disclosure from the vascular context into a retrieval system, and ultimately out of the patient.

As shown in FIGS. 6A-6F, an exemplary hooking mechanism 660 of the exemplary retrieval system 200 can be provided via the tubular structure 210 and controllable by a physician external to the patient, and configured to be radiopaque such that the physician can observe the manipulation through conventional fluoroscopic imaging. Once the hooking mechanism 660 is affixed to the hub 140 of the implanted prosthesis 100, as shown in FIG. 6B, the hooking mechanism 660 can be mechanically retracted, drawing the implanted prosthesis 100 towards the retrieval system 200 as shown in FIG. 6C. As the implant 100 is drawn towards the exemplary retrieval system 200, the implant 100 can begin to radially compress as shown in FIG. 6D, ultimately to a cross section suitable for enclosure as illustrated in FIGS. 6E and 6F. It is noted that procedures shown in FIGS. 6C and 6D can be interchanged in terms of an order of performance (e.g., more open, more closed, etc.)

A flow diagram of the method for a removal of the implant using the exemplary retrieval system 200 is shown in FIG. 3B. For example, in step 350, the exemplary procedure is initiated. In step 355, the prosthesis 100 has been deployed in a blood vessel or any other biological luminal structure. In step 365, it is determined whether the center ring 120 is still intact or mostly degraded. If not, the implant 100 is deemed to be implanted. However, if the center ring 120 is still intact/mostly degraded, the delivery vehicle 200 is used to navigate the implant in step 375. Then, in step 380, the retrieval/delivery vehicle 200 is extended. This can be done by e.g., by extending the connector 240 and the hooking arrangement 660, thereby opening up the clamps 220, as shown in FIG. 6B.

Thereafter, in step 385 of FIG. 3B, the implant 100 is collapsed into the retriever/delivery vehicle 200. This can be achieved by connecting the hooking arrangement 660 to the hub 140 of the implant, and pulling the connector 240 and the hooking arrangement 660, thereby closing the clamps 220 to envelop the contracted implant 100, the example of which is shown in FIGS. 6C-6D. This procedure then causes the implant 100 to contract, and be pulled within the tubular structure 210, as shown in FIGS. 6E-6F. In step 390, the retriever/delivery vehicle 200 is removed from the blood vessel or the luminal structure. Thus, in step 395, the implant 100 is removed from such blood vessel/luminal structure.

To improve and/or ensure a simple deconvolution of the three-space orientation and rotation of the implanted prosthesis 100, the exemplary implementation of the prosthesis 100 can include, e.g., a system of one or more radiopaque fiducials according to the exemplary embodiments of the present disclosure. Such exemplary one or more radiopaque fiducials can be fabricated by, e.g., ultrasonically coating a fifty-micron thick layer of silicone, designed to have an asymmetric pattern, with a layer of one hundred microns of tantalum. Other exemplary materials can be suitable for coating, or direct structural integration, including but not limited to, e.g., gold, palladium, platinum, iridium, cobalt chromium alloy, steel and steel alloys, nickel, tungsten, titanium, magnesium, or an alloy or combination of any two or more thereof. In some exemplary embodiments of the present disclosure, the fiducial can be biodegradable, and in other exemplary embodiments, the fiducial can be further encapsulated with a biocompatible material to isolate its radiopaque metallic surface from the patient.

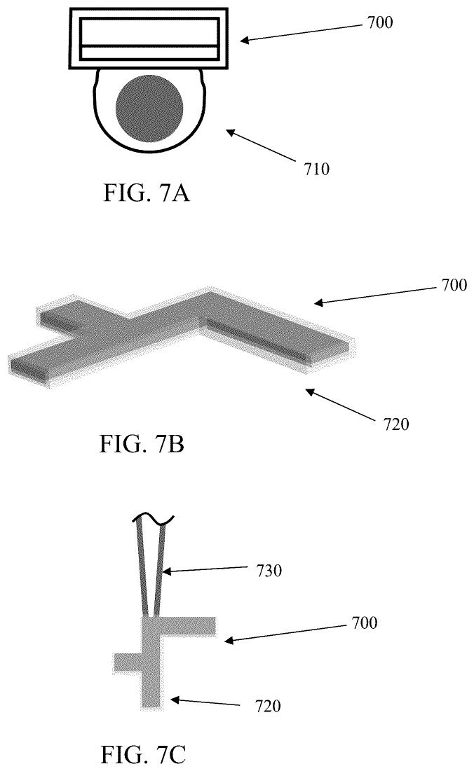

An exemplary illustration of a cross section 710 of an asymmetric imaging fiducial 700 that can be affixed to the exemplary vascular implant 100 and coated for isolation is shown in FIG. 7A. FIG. 7B illustrates a perspective view of the exemplary asymmetric fiducial 700 with an irregular shape 720. FIG. 7C shows a top view of the exemplary imaging fiducial 700 having the described irregular shape 720, and affixed to the vascular implant with exemplary connection(s) 730. Various exemplary designs can be achievable, whereas there is an asymmetry along one or more axes so as to provide different irregular shapes. In fact, the various exemplary designs discussed herein are not intended to limit the scope of the present disclosure in any manner. For example, according to further exemplary embodiments of the present disclosure, the exemplary fiducial 700 can be also be coated directly on one or more portions of the metallic scaffolding itself.

In the exemplary implementation, a flexible circuit board can contain electrodes

suitable for stimulating and recording tissue proximal to the prosthesis 100. The flexible interconnect(s) can be made from polyimide, parylene, liquid crystal polymer, or any flexible biocompatible material. The exemplary materials described herein are meant to be exemplary of preferred substrates, and are not intended to be limiting in any manner (indeed, other materials can be used). Electrodes can be fabricated directly on the flexible polymer using, e.g., conventional microfabrication techniques to deposit gold, platinum, iridium oxide metallic electrodes, or conductive polymer electrodes such as, for example, PEDOT:PSS. Electrode materials can also be transferred and/or bonded onto the flexible circuit board, such as, e.g., glassy carbon electrodes or platinum black electrodes. The exemplary flexible circuit board can include, e.g., in addition to the electrodes, microfabricated wire interconnects between distinct elements, such as the electrode system and active complementary metal-oxide semiconductor (CMOS) electronics.

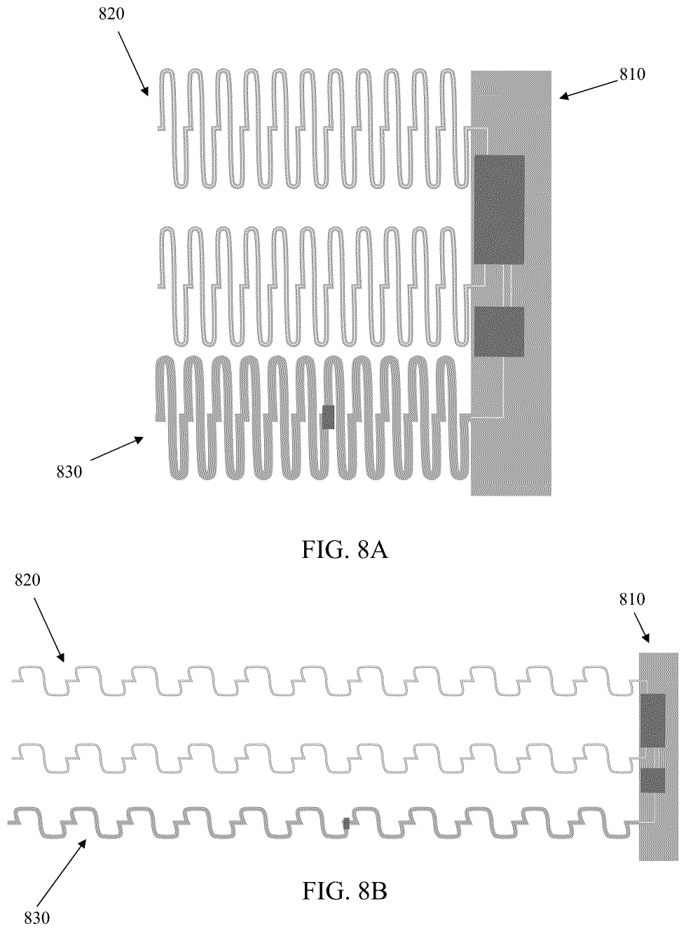

In the exemplary device according to the exemplary embodiments of the present disclosure of the present disclosure, the electrodes 820, 830 can be arrayed in a north-south zig-zag pattern, and interconnected with one another, e.g., via a connection device/configuration 810, as shown in FIG. 8A. In this exemplary manner, as the structural scaffolding expands from its radially compressed state to its radially expanded state, the array of the electrodes 820, 830 is able to stretch out to its extents and accommodate the circumferential increase of the prosthesis 100, as shown in FIG. 8B.

The exemplary electrode pattern can also accommodate a subsequent contraction from its radially expanded state back to its radially contracted state during re-sheathing. The same or similar north-south zig-zag pattern can be used to support interconnect to other elements of the implanted device, such as sensors or piezoelectrics, to facilitate a positioning of these elements at different rotational offsets along the circumference of the implanted device, as shown in the illustrations of FIGS. 8A and 8B. In the exemplary implementation, the expandable structural scaffolding can span the entire circumference of the implant 100 and the blood vessel/luminal structure. It is noted that there may be exemplary non-limiting scenarios in which the scaffolding should only extend to some percentage of the circumference of the entire device.

The exemplary implementation of the flexible circuit board according to the exemplary embodiments of the present disclosure can also include an active CMOS application specific integrated circuit (ASIC). The exemplary flexible circuit board can also include a wired interconnect patterned in an interfacial pattern matching the pad ring from the ASIC. The ASIC can then be flip chip bonded to the flexible circuit board using, e.g., conventional reflow balling and/or underfill techniques. In addition to the active electronics, the exemplary flexible circuit board can also include energy storage elements, such as, e.g., capacitors, and energy transduction elements, such as, e.g., piezoelectric transducers. The prosthesis 100 can include, in certain exemplary implementations, an antenna and/or other telemetry device on the flexible circuit board. The prosthesis 100 can also include, in certain exemplary implementations, a sensor array for measurement of biophysical signals of interest, including but not limited to pressure and/or temperature.

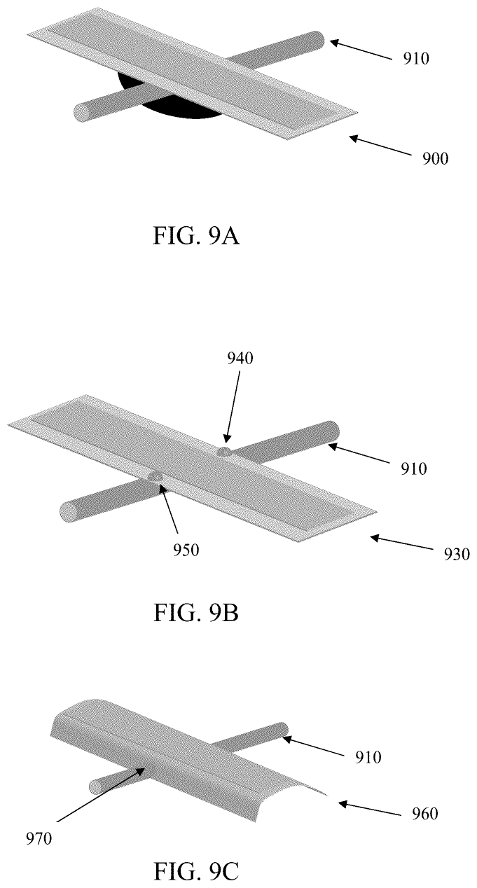

In an exemplary implementation, the flexible circuit board can be cut through laser micromachining using an excimer laser to release the zig-zag pattern of the electrodes 820, 830, facilitating a radial expansion and contraction. Regions of the flexible circuit board that do not directly facilitate interconnect and/or contain a functional element of the prosthesis can be cut away, thereby improving the porosity of the prosthesis. The patterned flexible circuit board can then be affixed to the structural scaffolding along each of the metallic support structures using a biocompatible adhesive. A representative illustration of a portion of an electrode 900 affixed to a strut 910 is shown in FIG. 9A. Alternatively or in addition, a sheet of the a flexible electrode 930 can accommodate mounting points 940, 950 that correspond to rivet-like structures on the structural support, as shown in FIG. 9B. Further exemplary alternatives can include, e.g., laser micromachining structural support holes 970 that match the metallic scaffolding may be cut into the package such that the struts run through portions 960 of the flexible circuit board, entering at a proximal point and exiting at a distal point, as shown in the illustration of FIG. 9C. This can facilitate a tighter integration between the flexible circuit board and the wire-mesh support structure, while ensuring the electrode is in tight apposition with the vessel wall.

In alternative exemplary implementations, the flexible circuit board can be first patterned with laser micromachining or any other etching technique. Still further implementations can facilitate for disparate aspects of the structural patterning to occur at various points over the complete fabrication cycle. The exemplary implementation is not intended to restrict the present disclosure to the specified order of fabrication steps described herein, and merely provides one exemplary way to implement certain exemplary embodiments of the present disclosure.

Throughout the disclosure, the following terms take at least the meanings explicitly associated herein, unless the context clearly dictates otherwise. The term “or” is intended to mean an inclusive “or.” Further, the terms “a,” “an,” and “the” are intended to mean one or more unless specified otherwise or clear from the context to be directed to a singular form.

In this description, numerous specific details have been set forth. It is to be understood, however, that implementations of the disclosed technology can be practiced without these specific details. In other instances, well-known methods, structures and techniques have not been shown in detail in order not to obscure an understanding of this description. References to “some examples,” “other examples,” “one example,” “an example,” “various examples,” “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described may include a particular feature, structure, or characteristic, but not every implementation necessarily includes the particular feature, structure, or characteristic. Further, repeated use of the phrases “in one example,” “in one exemplary embodiment,” or “in one implementation” does not necessarily refer to the same example, exemplary embodiment, or implementation, although it may.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain implementations of the disclosed technology, including the best mode, and also to enable any person skilled in the art to practice certain implementations of the disclosed technology, including making and using any devices or systems and performing any incorporated methods. The patentable scope of certain implementations of the disclosed technology is defined in the claims, and may include other examples that occur to those skilled in the art. Such other examples are intended to be within the scope of the claims if they have structural elements that do not differ from the literal language of the claim, or if they include equivalent structural elements with insubstantial differences from the literal language of the claims.

### EXEMPLARY REFERENCES

The following reference is hereby incorporated by references in their entireties:
1. Cracchiolo, M. et al., “Bioelectronic medicine for the autonomic nervous system: clinical applications and perspectives.” J Neural Eng 18 (4), (2021).
2. Oxley, T. J. et al., “Minimally invasive endovascular stent-electrode array for high-fidelity, chronic recordings of cortical neural activity.” Nature Biotechnology 34 (3): 320-327 (2016).

1. An intravascular prosthesis, comprising:
an expandable configuration that is configured or structured to be inserted within a luminal biological structure, the expandable configuration comprising:
at least one self-expanding system which is (i) at least one of a wire system or a mesh system, and (ii) configured to include a plurality of apical connectors, with a subset thereof being connected to a medial structure, and
at least one flexible interconnect system configured or structured to house or provide therein or thereon (i) at least one integrated circuit, (ii) at least one electrode, and (iii) at least one transducer.

2. The intravascular prosthesis of claim 1, wherein the expandable configuration is at least one of:
(a) further configured or structured to be deployed with a delivery vehicle into the luminal biological structure by (i) compressing the housing to form a compressed state, and (ii) deploying the expandable configuration at a location of a therapeutic relevance by expanding the housing from the compressed state to an expanded state at one or more extents of the luminal biological structure, or
(b) configured or structured to include an asymmetric fluoroscopic imaging fiducial

3. The intravascular prosthesis of claim 2, wherein the luminal biological structure is a blood vessel.

4. The intravascular prosthesis of claim 1, wherein the flexible interconnect system is at least one flexible circuit board.

5. The intravascular prosthesis of claim 4, wherein the at least one flexible circuit board comprises polyimide and metal interconnects.

6. The intravascular prosthesis of claim 4, wherein the at least one electrode at least one of:
(a) spans a predetermined percentage of a dimension of the at least one flexible circuit board, such that (i) when compressed the at least one flexible circuit board maintains a substantially similar cross-sectional area as that of a mesh scaffolding, and (ii) when expanded in the blood vessel, the at least one flexible circuit board spans a predetermined percentage of a circumference of the luminal biological structure, or
(b) is configured to stimulate or record electrophysiological information for at least one biological portion provided in or at the luminal biological structure.

7. (canceled)

8. The intravascular prosthesis of claim 1, wherein the at least one self- expanding system at least one of (i) includes thereon or therein a pharmacological-eluting coating, or (ii) includes the medial structure.

9. (canceled)

10. The intravascular prosthesis of claim 1, wherein the at least one transducer includes a piezoelectric material that is configured to provide telemetry and power to the at least one integrated circuit with ultrasound.

11. The intravascular prosthesis of claim 1, wherein the medial structure is configured to at least one of (i) facilitate a retrieval of the intravascular prosthesis from a patient, (ii) dissolve to nontoxic species over a controlled time frame, or (iii) facilitate a placement of the intravascular prosthesis into a patient.

12.-14 (canceled)

15. The intravascular prosthesis of claim 1, wherein the medial structure is a hub.

16. The intravascular prosthesis of claim 14, wherein the at least one self-expanding system is an expandable housing which includes the medial structure.

17. An intravascular prosthesis, comprising:
an expandable configuration that is configured or structured to be inserted within a luminal biological structure, the expandable configuration comprising:
at least one self-expanding system which is (i) at least one of a wire system or a mesh system, and (ii) configured to include a plurality of apical connectors, and
a medial structure connected to one or more of the apical connectors, wherein the medial structure is configured to (i) facilitate a placement of the intravascular prosthesis into a patient, and (ii) be dissolved within the luminal biological structure to nontoxic species over a controlled time frame.

18. The intravascular prosthesis of claim 17, wherein the medial structure is at least one of (i) configured to facilitate a retrieval of the intravascular prosthesis from a patient, (ii) configured to facilitate a placement of the intravascular prosthesis into a patient, or (iii) a hub.

19-20. (canceled)

21. The intravascular prosthesis of claim 17, wherein the at least one self-expanding system at least one of (i) is an expandable housing which includes the medial structure, or (ii) include thereon or therein a pharmacological-eluting coating.

22. The intravascular prosthesis of claim 17, wherein the expandable configuration at least one of:
(a) further comprises at least one flexible interconnect system configured or structured to house or provide therein or thereon (i) at least one integrated circuit, (ii) at least one electrode, and (iii) at least one transducer,
(b) is further configured or structured to be deployed with a delivery vehicle into the luminal biological structure by (i) compressing the housing to form a compressed state, and (ii) deploying the expandable configuration at a location of a therapeutic relevance by expanding the housing from the compressed state to an expanded state at one or more extents of the luminal biological structure, or
(c) is configured or structured to include an asymmetric fluoroscopic imaging fiducial.

23. (canceled)

24. The intravascular prosthesis of claim 22, wherein the luminal biological structure is a blood vessel.

25. The intravascular prosthesis of claim 22, wherein the flexible interconnect system is at least one flexible circuit board.

26. The intravascular prosthesis of claim 25, wherein the at least one flexible circuit board comprises polyimide and metal interconnects.

27. The intravascular prosthesis of claim 25, wherein the at least one electrode at least one of:
(a) spans a predetermined percentage of a dimension of the at least one flexible circuit board, such that (i) when compressed the at least one flexible circuit board maintains a substantially similar cross-sectional area as that of a mesh scaffolding, and (ii) when expanded in the blood vessel, the at least one flexible circuit board spans a predetermined percentage of a circumference of the luminal biological structure, or
(b) is configured to stimulate or record electrophysiological information for at least one biological portion provided in or at the luminal biological structure.

28-30. (canceled)

31. The intravascular prosthesis of claim 23, wherein the at least one transducer includes a piezoelectric material that is configured to provide telemetry and power to the at least one integrated circuit with ultrasound.

32. A method for installing an intravascular prosthesis within a luminal biological structure, comprising:
providing a tubular device into the luminal biological structure;
extending the intravascular prosthesis which includes an expandable configuration through the tubular device via an implantation device, the expandable configuration comprising:
at least one self-expanding system which is (i) at least one of a wire system or a mesh system, and (ii) includes a plurality of apical connectors, and
at least one flexible interconnect system configured or structured to house or provide therein or thereon (i) at least one integrated circuit, (ii) at least one electrode, and (iii) at least one transducer;
pushing the intravascular prosthesis into the luminal biological structure via the implantation device to be outside of the tubular device so that the self-expanding system opens by expanding the plurality of apical connectors to contacts inner walls of the luminal biological structure; and
disconnecting the implantation device from the intravascular prosthesis to implant the intravascular prosthesis, and removing the tubular device and the implantation device from the biological structure.

33. A method for installing an intravascular prosthesis within a luminal biological structure, comprising:
providing a tubular device into the luminal biological structure;
extending the intravascular prosthesis which includes an expandable configuration through the tubular device via an implantation device, the expandable configuration comprising:
at least one self-expanding system which is (i) at least one of a wire system or a mesh system, and (ii) includes a plurality of apical connectors, and
a medial structure connected to one or more of the apical connectors, and which is be dissolved within the tubular biological structure to nontoxic species over a controlled time frame;
pushing the intravascular prosthesis into the luminal biological structure via the implantation device to be outside of the tubular device so that the self-expanding system opens by expanding the plurality of apical connectors to contacts inner walls of the luminal biological structure; and
disconnecting the implantation device from the intravascular prosthesis to implant the intravascular prosthesis, and removing the tubular device and the implantation device from the biological structure.
